SEQUENCE LISTING (35 sequences)

SEQ ID NO: 1 - protein, 5 aa, Synthetic construct
  source 1..5: note = Linker
GGGGS

SEQ ID NO: 2 - protein, 10 aa, Synthetic construct
  source 1..10: note = linker
GGGGSGGGGS

SEQ ID NO: 3 - protein, 15 aa, Synthetic construct
  source 1..15: note = linker
GGGGSGGGGSGGGGS

SEQ ID NO: 4 - protein, 15 aa, Synthetic construct
  source 1..15: note = linker
EAAAKEAAAKEAAAK

SEQ ID NO: 5 - protein, 45 aa, Synthetic construct
  source 1..45: note = linker
NGLLIPQFLVASGGGGSRSIVTFSLRQNRGGGGSNGLLIPQFLAS

SEQ ID NO: 6 - protein, 49 aa, Synthetic construct
  source 1..49: note = linker
NGLLIPQFLVASGGGGSRALQALRALQALEALGGGGSNGLLIPQFLVAS

SEQ ID NO: 7 - protein, 49 aa, Synthetic construct
  source 1..49: note = linker
NGLLIPQFLVASGGGGSRALRALQALEALEALGGGGSNGLLIPQFLVAS

SEQ ID NO: 8 - protein, 12 aa, Synthetic construct
  source 1..12: note = linker
RSIVTFSLRQNR

SEQ ID NO: 9 - protein, 15 aa, Synthetic construct
  source 1..15: note = linker
RALQALRALQALEAL

SEQ ID NO: 10 - protein, 15 aa, Synthetic construct
  source 1..15: note = linker
RALRALQALEALEAL

SEQ ID NO: 11 - protein, 25 aa, Synthetic construct
  source 1..25: note = linker
NGLLIPQFLVASGGGGSMSDYQMDM

SEQ ID NO: 12 - protein, 28 aa, Synthetic construct
  source 1..28: note = linker
RALRALQALEALEALGGGGSMSDYQMDM

SEQ ID NO: 13 - protein, 28 aa, Synthetic construct
  source 1..28: note = linker
RALQALRALQALEALGGGGSMSDYQMDM

SEQ ID NO: 14 - protein, 27 aa, Synthetic construct
  source 1..27: note = linker
NGLLIPQFLVASGGGGSMDMQGRYMDR

SEQ ID NO: 15 - protein, 30 aa, Synthetic construct
  source 1..30: note = linker
RALRALQALEALEALGGGGSMDMQGRYMDR

SEQ ID NO: 16 - protein, 30 aa, Synthetic construct
  source 1..30: note = linker
RALQALRALQALEALGGGGSMDMQGRYMDR

SEQ ID NO: 17 - protein, 64 aa, Synthetic construct
  source 1..64: note = artificial peptide composed of at least a textile binding domain and a chlorine scavenger domain
MYMDMKDVMMKYDMAMMEMKMDSGGGRSIVTFSLRQNRGGGSMYMDMKDVMMKYDMAMMEMKMD

SEQ ID NO: 18 - protein, 56 aa, Synthetic construct
  source 1..56: note = artificial peptide composed of at least a textile binding domain and a chlorine scavenger domain
MYMDMKDVMMKYDMAMMEMKMDRSIVTFSLRQNRMYMDMKDVMMKYDMAMMEMKMD

SEQ ID NO: 19 - protein, 28 aa, Synthetic construct
  source 1..28: note = artificial peptide composed of at least a textile binding domain and a chlorine scavenger domain
MKMYMKMDRSIVTFSLRQNRMKMYMKMD

SEQ ID NO: 20 - protein, 26 aa, Synthetic construct
  source 1..26: note = artificial peptide composed of at least a textile binding domain and a chlorine scavenger domain
MMYMMYMRSIVTFSLRQNRMMYMMYM

SEQ ID NO: 21 - protein, 46 aa, Synthetic construct
  source 1..46: note = artificial peptide composed of at least a textile binding domain and a chlorine scavenger domain
NGLLIPQFLVASGGGGSRSIVTFSLRQNRGGGGSNGLLIPQFLVAS

SEQ ID NO: 22 - protein, 64 aa, Synthetic construct
  source 1..64: note = artificial peptide composed of at least a textile binding domain and a chlorine scavenger domain
KYGDMKDVKMKYDKACSEMKYDSGGGRSIVTFSLRQNRGGGSKYGDMKDVKMKYDKACSEMKYD

SEQ ID NO: 23 - protein, 56 aa, Synthetic construct
  source 1..56: note = artificial peptide composed of at least a textile binding domain and a chlorine scavenger domain
KYGDMKDVKMKYDKACSEMKYDRSIVTFSLRQNRKYGDMKDVKMKYDKACSEMKYD

SEQ ID NO: 24 - protein, 5 aa, Synthetic construct
  source 1..5: note = linker
GGGGS

SEQ ID NO: 25 - protein, 10 aa, Synthetic construct
  source 1..10: note = linker
GGGGSGGGGS

SEQ ID NO: 26 - protein, 15 aa, Synthetic construct
  source 1..15: note = linker
GGGGSGGGGSGGGGS

SEQ ID NO: 27 - protein, 20 aa, Synthetic construct
  source 1..20: note = linker
GGGGSGGGGSGGGGSGGGGS

SEQ ID NO: 28 - protein, 8 aa, Synthetic construct
  source 1..8: note = linker
GGGGGGGG

SEQ ID NO: 29 - protein, 6 aa, Synthetic construct
  source 1..6: note = linker
GGGGGG

SEQ ID NO: 30 - protein, 5 aa, Synthetic construct
  source 1..5: note = linker
EAAAK

SEQ ID NO: 31 - protein, 10 aa, Synthetic construct
  source 1..10: note = linker
EAAAKEAAAK

SEQ ID NO: 32 - protein, 15 aa, Synthetic construct
  source 1..15: note = linker
EAAAKEAAAKEAAAK

SEQ ID NO: 33 - protein, 46 aa, Synthetic construct
  source 1..46: note = linker
AEAAAKEAAAKEAAAKEAAAKALEAEAAAKEAAAKEAAAKEAAAKA

SEQ ID NO: 34 - protein, 5 aa, Synthetic construct
  source 1..5: note = linker
PAPAP

SEQ ID NO: 35 - protein, 12 aa, Synthetic construct
  source 1..12: note = linker
AEAAAKEAAAKA

(19) United States
(12) Patent Application Publication
(10) Pub. No.: US 2026/0028556 A1
(43) Pub. Date: Jan. 29, 2026

(54) USE OF PEPTIDES IN FABRIC FINISHING FORMULATIONS FOR COLOR CARE

(71) Applicant: Henkel AG & Co. KGaA, Duesseldorf (DE)

(72) Inventors: Daniel Thomas Piorkowski, Fairfield, CT (US); Christian Degering, Erkrath (DE); Casey Elphege Camire, Trumbull, CT (US); Meghan Russell, Monroe, CT (US); Steven Rodriguez, Cheshire, CT (US); Kyle Seamus Joseph O'Connor, Stamford, CT (US); Fabian Falkenberg, Neuss (DE)

(21) Appl. No.: 19/258,463

(22) Filed: Jul. 2, 2025

Related U.S. Application Data
(60) Provisional application No. 63/674,518, filed on Jul. 23, 2024.

(51) Int. Cl. C11D 3/38; C02F 1/68; C02F 101/12; C07K 14/00; C11D 3/30; C11D 3/43; D06F 35/00

(52) U.S. Cl. CPC C11D 3/38; C02F 1/683; C07K 14/001; C11D 3/30; C11D 3/43; D06F 35/006; C02F 2101/12; C02F 2307/12

(57) ABSTRACT

Disclosed are color care compositions including liquid rinse aid compositions comprising a peptide capable of scavenging chlorine. The peptide can further have textile binding capabilities to provide carryover benefits from a wash cycle to a rinse cycle.

### CROSS-REFERENCE TO RELATED APPLICATION

This application claims priority to and the benefit of U.S. Provisional Patent Application No. 63/674,518, filed on Jul. 23, 2024. The related application is incorporated herein by reference in its entirety.

### SEQUENCE LISTING

The Instant Application contains a Sequence Listing which has been submitted electronically in XML format and is hereby incorporated by reference in its entirety. Said XML copy, created on Jun. 24, 2025, is named “HEN0009US2” and is 38,542 bytes in size. The Sequence Listing does not go beyond the disclosure in the application as filed.

### BACKGROUND

Water sourced from municipalities are typically made potable by the introduction of chlorine as a sanitization agent against microbes. Although rendered potable, municipal water treated with chlorine contains residual levels of chlorine. This same water is used for domestic cleaning, including laundry. Chlorine is known to deteriorate dyes found in fabrics over time, especially red, blue, and black dyes. If the chlorine content in water is not controlled, dye deterioration from fabrics may be seen in 3 to 30 washes. In addition to fabric dye damage and color loss, failing to control chlorine in the water can negatively impact enzyme performance, for example in detergents employing enzymes for cleaning.

Even if chlorine were to be scavenged during a laundry wash cycle, the same municipal water containing chlorine will be used in a rinse cycle, thereby reintroducing chlorine to the fabrics and textiles being laundered. Although some fabric softeners, which are added in the rinse cycle, can scavenge chlorine, not all consumers use fabric softeners and not all fabrics are compatible with fabric softeners.

There remains a need in the art for laundry care products that can remove chlorine from both the wash water in the wash cycle and the rinse water in the rinse cycle.

### SUMMARY

Disclosed, in various embodiments, is a liquid color care rinse aid composition, comprising an artificial peptide; a liquid diluent; and optionally an additional ingredient, wherein the artificial peptide is composed of a textile binding domain and a chlorine scavenger domain.

In another embodiment, a method of scavenging chlorine from a wash cycle and a rinse cycle comprises contacting a fabric or textile with a liquid color care composition in a laundry wash cycle, rinse cycle, or a combination thereof, wherein the liquid color care composition comprises an artificial peptide; a liquid diluent; and optionally an additional ingredient, wherein the artificial peptide is composed of a textile binding domain and a chlorine scavenger domain.

In another embodiment, a method of preventing the deterioration of dyes in a fabric or textile comprises contacting a fabric or textile with a liquid color care composition in a laundry wash cycle, rinse cycle, or a combination thereof, wherein the liquid color care composition comprises an artificial peptide; a liquid diluent; and optionally an additional ingredient, wherein the artificial peptide is composed of a textile binding domain and a chlorine scavenger domain.

In another embodiment, a method of preventing color loss from a fabric or textile, comprises contacting a fabric or textile with a liquid color care composition in a laundry wash cycle, rinse cycle, or a combination thereof, wherein the liquid color care composition comprises an artificial peptide; a liquid diluent; and optionally an additional ingredient, wherein the artificial peptide is composed of a textile binding domain and a chlorine scavenger domain.

These and other features and characteristics are more particularly described below.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Disclosed herein are peptides comprising amino acid groups capable of scavenging chlorine, and color care and fabric rinse aid compositions comprising these peptides.

Peptides containing chlorine scavenging amino acids such as Lysine and Threonine can provide liquid color care fabric rinse aid compositions with the benefit of scavenging chlorine. The chlorine scavenging ability of these peptides provide fabric rinse aid compositions that can provide benefits such as the ability to scavenge chlorine from the water used in the wash cycle, prevent deterioration of fabric dye over repeated washings, and/or improve or maintain the cleaning performance of detergents containing enzymes as the peptide will remove chlorine from the wash water, leaving less chlorine to interact with performance enzymes (which deactivates them).

The peptides described herein are further engineered to adhere to various textiles, so that the peptides can carry over to the rinse cycle and scavenge chlorine in the rinse water. This carryover will provide a color care benefit, since rinse aid added to the wash will only scavenge chlorine in the wash, leaving textiles susceptible to chlorine in the rinse water. Although products such as fabric softeners containing ester quats will scavenge chlorine in the rinse since they are added to the rinse cycle but not all consumers use fabric softeners. The use of the specialized peptide described herein will provide the consumer with the benefit of scavenging chlorine in both the wash and rinse cycles, providing better color care to their fabrics and textiles.

Certain peptides and/or peptide conjugates with a hydrophobic end and a hydrophilic end are particularly noteworthy here, in which the hydrophobic part can bind to the textile and the hydrophilic part cannot. Particularly advantageous peptides can adhere to plastic surfaces, specifically LSEP (low surface energy polymers) surfaces and in particular textiles made of (LSEP) plastic or with a (LSEP) plastic content. For example, peptides with strong alpha-helices and/or high arginine content are foreseen to be good polyester-binders. Therefore, these peptides and/or peptide conjugates are particularly suitable for use in color care rinse aid compositions to give the textiles special properties. When these peptides contain chlorine scavenging amino acids, they can bind to textiles and provide color care properties in rinse cycle as well as subsequent wash and rinse cycles.

In certain embodiments, a peptide sequence encoding a peptide chain may be divided into one or more domains, for example, with a “textile binding” domain, a “linker” domain, and a “chlorine scavenger” domain. Thus, embodiments of the present disclosure may fit into the overall form of B-L-S where B is a textile binding domain, L is a linker domain, and S is a chlorine scavenger domain.

In still other embodiments one or more additional peptide sequences may encode one or more additional peptide chains that may be cross-linked or bridged (e.g., by using one or more small molecules, polymers, cross-linkers, etc.) to a first peptide chain. The one or more additional chains may be additional linker, textile binder, or chlorine scavenger domains. Or, in still other embodiments, the additional peptide chain may contain functional groupings for enhanced soil removal, fabric care, ingredient stabilization, and the like.

A “peptide” in the context of the present disclosure is to be understood as meaning a polymer composed of amino acids, specifically the 20 proteinogenic L-amino acids, specifically of linear structure, which has up to 100 amino acids which are linked to one another via peptide bonds. According to the disclosure, the peptides of the disclosure have an amino acid sequence of 4 to 80 amino acids, specifically 10 to 60. In certain embodiments further described herein, a linker sequence may form a part of the total amino acid count. In still other embodiments the amino acid count is reflective of the non-linker portion or portion. In the context of this disclosure, the amino acids are given in a one-letter code, where, for example, C stands for cysteine, R for arginine, A for alanine and L for leucine. It is further understood that unless otherwise indicated, the amino acids in an amino acid sequence disclosed herein are linked via peptide bonds and, unless otherwise indicated, the sequence is listed in N- to C-terminal orientation.

Peptides can be chemically synthesized in various embodiments and/or recombinantly produced using protein design. Short peptides can easily be prepared synthetically, for example via solid phase synthesis. Longer peptides and polypeptides, on the other hand, are often produced recombinantly in A host organism.

Typical acidic or negatively charged amino acids (depending on pH) are D and E.

The positively charged or basic amino acids (depending on the pH value) typically include R, K and H.

Amino acids such as G, A, C, I, L, M, F, V, P, S, T, W, Y, N and Q are typically uncharged, i.e. neutral, amino acids.

When reference is made herein to an “any” amino acid, what is commonly meant is one of the 20 naturally occurring proteinogenic amino acids, i.e. one of glycine (G), alanine (A), valine (V), leucine (L), isoleucine (I), Phenylalanine (F), Serine (S), Threonine (T), Proline (P), Methionine (M), Cysteine (C), Histidine (H), Lysine (K), Arginine (R), Glutamine (Q), asparagine (N), aspartic acid (D), glutamic acid (E), tyrosine (Y), and tryptophan (W). Unless otherwise stated, the amino acids are typically L-amino acids. In alternative embodiments, the peptide can also consist of D-amino acids, although it may be that D- and L-amino acids do not occur at the same time within the peptides described herein. In various embodiments, any such amino acid includes all of the aforementioned amino acids with the exception of proline, or in some embodiments also with the exception of proline and glycine. These two amino acids are not in certain embodiments because they have helix-breaking properties and can therefore adversely affect the secondary structure of the peptides.

In certain embodiments, the peptide has a total charge of −2 to +12, specifically 0 to +8, more specifically 0 to +4, particularly specifically 0 to +2. The total charge of the peptide is based on the number of positively and negatively charged amino acids in the peptide, in particular arginine (R), lysine (K), histidine (H), aspartic acid (D) and glutamic acid (E) and is the sum of the negative ones and positive charges, whereby one positive and one negative charge cancel each other out. A peptide with 2 arginine residues and 1 glutamic acid residue therefore had a total charge of +1. The total charge of the peptide is specifically −2 to +12, more specifically 0 to +8, particularly specifically 0 to +4, very particularly specifically 0 to +2.

In the context of the textile binding domain, the peptide specifically comprises amino acids with a high alpha-helix-forming potential, these amino acids being selected from E, A, L, M, Q, K, R, F, I, H, W and D, more specifically from E, A, L, M, Q, K, R, F, I and H; particularly specifically from E, A, L, M, Q, K, R and F.

In certain embodiments, the peptide is at least 40%, 45%, 50%, 55%, or 60% and increasingly specifically at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94% or 95% of amino acids with a high alpha-helix-forming potential, these amino acids being specifically selected from E, A, L, M, Q, K, R, F, I, H, W and D, more specifically E, A, L, M, Q, K, R, F, I and H; particularly specifically from E, A, L, M, Q, K, R and F.

The peptide according to the disclosure particularly forms a helical secondary structure, in particular an alpha-helix structure, specifically with an alpha-helix content of at least 80% and increasingly specifically of at least 81%, 82%, 83%, 84%, 85%, 86%, 87%, 88%, 89%, 90%, 91%, 92%, 93%, 94% or 95%, especially higher than 95%. The use of the motif AL or LA in the amino acid sequence of the peptide according to the disclosure can contribute to the stability of the helix structure because these amino acids have a high alpha-helix-forming potential. The alpha-helix structure is particularly suited for the textile binding domain of the peptide.

Textiles containing plastics, or mixtures of natural fibers and plastics are suitable for adhesion. By way of non-limiting examples, suitable textiles for use with the present disclosure can include, polyesters, polyester-containing blended fabrics (in particular, polyester/cotton blended fabrics). Other fibers (linens, hemp, silk, etc.) and plastics (nylon, rayon, etc.) are also contemplated and capable of adhesion. Peptides that demonstrate good adhesion, have an amino acid sequence that is at least 80% and increasingly specifically at least 81%, 82%, 83%, 84%, 85%, 86%, 87%, 88%, 89%, 90%, 91%, 92%, 93%, 94%, 95%, 96%, 97%, 98%, 99% or 100% sequence identity to one of the in SEQ ID NO:1-32 have the amino acid sequences specified.

Methods for determining adhesion are known to those skilled in the art and all suitable methods can be used.

A common method for determining adhesion is based on a direct measurement method with BCA (bicinchoninic acid; 2,2′-biquinolyl-4,4′-dicarboxylic acid). To do this, cloth flaps are mixed with a peptide solution to be tested (0.02 mg mL⁻¹ in distilled water), incubated for 1 hour at room temperature shaking (750 rpm) and washed (3× with 0.5 mL distilled water, shaking at 750 rpm for 5 min), then 0.2 ml of distilled water are added and 0.2 ml BCA reagent was added and incubated for 60 min at 60° C. The absorption is then measured at 562 nm. An adhesion point (AP) after an infinite adhesion time can be read from the measured adhesion values. AP in % shows how much % of the applied amount (100%) remains on the cloth after washing. The higher the AP value, the higher the binding affinity and thus the adhesion. (<20%=no adhesion; 21 to 40%=low adhesion; 41 to 60%=medium adhesion; 61 to 80%=good adhesion; >80%=very good adhesion).

For the purposes of the disclosure, a peptide is a textile-binding peptide if the ability of the peptide to adhere to textile surfaces is demonstrated in either the herein-described method or in one used in the art. The common theme is the binding affinity being greater than that of a reference molecule and the textile-binding peptides described herein specifically being one 10-fold, more specifically 20-fold, 50-fold or 100-fold greater adhesion to a given surface than any alternative peptide of comparable length that was not designed for this purpose and does not meet the sequence specifications described herein.

In various embodiments of the disclosure at least one, specifically at least two, textile-non-binding peptide(s) is directly covalently linked to the at least one textile-binding peptide, i.e. the first and/or last amino acid. The respective peptides are linked to each other via a peptide bond. Alternatively, the binding can also take place via a linker, in particular a peptide linker. Suitable linkers are known in the art and can be static/stiff or flexible. This property is determined by the secondary structure of the linker, for example stiff linkers can have an alpha helix as the secondary structure. In various embodiments, the peptide linker sequence is flexible and has no secondary structure or only short secondary structure elements. Linkers suitable in the context of the present disclosure are described below.

Linkers (occasionally known as “spacers”) suitable in the context of the present disclosure, which are specifically peptide linkers, can be divided into flexible linkers and stiff linkers. Such linkers are basically known in the art (Chen et al. (2013) Fusion protein linkers: Property, design and functionality, Advanced Drug Delivery Reviews, 65 (10): 1357-1369).

Typically, such peptide linkers have a length of 1 to 200 amino acids, for example 1 to 100 amino acids, specifically 2 to 30 amino acids, more specifically 5 to 25 amino acids.

In various embodiments, the linker is selected from the following group:

(I)
(GGGGS)ₙ with n = 1, 2, 3, or 4;
(II)
(G)₆;
(III)
(G)₈;
(IV)
(EAAAK)ₙ with n = 1, 2, or 3;
(V)
A(EAAAK)₄ALEA(EAAAK)₄A;
(VI)
PAPAP;
(VII)
AEAAAKEAAAKA;
and
(VIII)
(AP)ₙ with n = 10-34.

In the context of the present disclosure, functional homologs of the aforementioned linker sequences are also suitable. “Functional homologues” as used in this context refers to sequences that are at least 80% and increasingly specifically at least 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 98.8%, 99.0%, 99.2%, 99.4% or 99.6% identical to that are the specified reference sequence and have their functionality, i.e. can act as a bond between peptides and/or peptide and a PEG chain, without impairing the advantageous properties of corresponding peptide conjugates, as described and disclosed herein.

Linkers according to the invention have one of the following amino acid sequences:

(SEQ ID NO: 24)
GGGGS,
(SEQ ID NO: 25)
GGGGSGGGGS,
(SEQ ID NO: 26)
GGGGSGGGGSGGGGS,
(SEQ ID NO: 27)
GGGGSGGGGSGGGGSGGGGS,
(SEQ ID NO: 28)
GGGGGGGG,
(SEQ ID NO: 29)
GGGGGG,
(SEQ ID NO: 30)
EAAAK,
(SEQ ID NO: 31)
EAAAKEAAAK,
(SEQ ID NO: 32)
EAAAKEAAAKEAAAK,
(SEQ ID NO: 33)
AEAAAKEAAAKEAAAKEAAAKALEAEAAAKEAAAKEAAAKEAAAKA,
(SEQ ID NO: 34)
PAPAP
or
(SEQ ID NO: 35)
AEAAAKEAAAKA.

The linker with the following amino acid sequence is particularly useful:

(SEQ ID NO: 26)
GGGGSGGGGSGGGGS
or
(SEQ ID NO: 32)
EAAAKEAAAKEAAAK.

The identity of nucleic acid or amino acid sequences is determined by sequence comparison. This sequence comparison is based on the BLAST algorithm established and commonly used in the art (cf. e.g. Altschul et al. (1990) Basic local alignment search tool, J. Mol. Biol., 215:403-410, and Altschul et al. (1997) Gapped BLAST and PSI-BLAST: a new generation of protein database search programs, Nucleic Acids Res., 25:3389-3402) and basically happens by similar sequences of nucleotides or amino acids in the nucleic acid or amino acid sequences be assigned. A tabular assignment of the relevant positions is called alignment. Another algorithm available in the art is the FASTA algorithm. Sequence comparisons (alignments), especially multiple sequence comparisons, are created using computer programs. For example, the Clustal series (see e.g. Chenna et al. (2003) Multiple sequence alignment with the Clustal series of programs, Nucleic Acid Res., 31:3497-3500), T-Coffee (see e.g. Notredame et al. (2000) T-Coffee: A novel method for multiple sequence alignments, J. Mol. Biol., 302:205-217) or programs based on these programs or algorithms. Sequence comparisons (alignments) are also possible using the computer program Vector NTI® Suite 10.3 (Invitrogen Corporation, 1600 Faraday Avenue, Carlsbad, California, USA) with the specified standard parameters, whose AlignX module for sequence comparisons is based on ClustalW, or Clone Manager 10 (Use of the BLOSUM 62 scoring matrix for sequence alignment at the amino acid level). Unless otherwise stated, sequence identity reported herein is determined using the BLAST algorithm.

Such a comparison also allows a statement to be made about the similarity of the compared sequences to one another. It is usually given as percent identity, i.e. the proportion of identical nucleotides or amino acid residues in the same positions or in positions corresponding to one another in an alignment. The broader concept of homology includes conserved amino acid exchanges in amino acid sequences, i.e. amino acids with similar chemical activity, since these usually exert similar chemical activities within the protein. Therefore, the similarity of the compared sequences can also be stated as percent homology or percent similarity. Identity and/or homology information can be made for entire polypeptides or genes or just for individual regions. Homologous or identical regions of different nucleic acid or amino acid sequences are therefore defined by matches in the sequences. Such areas often have identical functions. They can be small and contain only a few nucleotides or amino acids. Such small areas often perform essential functions for the overall activity of the protein. It can therefore make sense to relate sequence matches only to individual, possibly small areas. Unless otherwise stated, identity or homology information in the present application refers to the total length of the nucleic acid or amino acid sequence specified in each case.

The peptide or protein concentration can be determined using known methods, for example the BCA method (bicinchoninic acid; 2,2′-biquinolyl-4,4′-dicarboxylic acid) or the biuret method (Gornall et al., J. Biol. Chem., 1948, 177:751-766). Those skilled in the art of peptide and protein technology will be aware of a variety of suitable methods for determining peptide or protein concentration that can be used within the scope of this disclosure.

In certain embodiments of the disclosure, the peptide may be present from 0.00001 to 1 wt. %, such as in an amount of from 0.0001 to 0.5 wt. %, or in an amount of from 0.001 to 0.1 wt. %, in each case based on the active peptide.

Peptides according to the disclosure can have amino acid changes, in particular amino acid substitutions, insertions or deletions. Such peptides are further developed, for example, through targeted genetic modification, i.e. through mutagenesis processes, and optimized for specific purposes or with regard to special properties (e.g. in terms of their stability, binding, etc.).

For example, targeted mutations such as substitutions, insertions or deletions can be introduced into the known molecules in order to change certain properties, for example. For this purpose, in particular the surface charges and/or the isoelectric point of the molecules and thereby their interactions with a surface can be changed. For example, the net charge of the peptides can be changed in order to influence substrate binding. Alternatively, or additionally, one or more corresponding mutations can, for example, increase the stability or adsorption of the peptide. Advantageous properties of individual mutations, e.g. individual substitutions, can complement each other.

The disclosure therefore also includes peptides which are characterized in that they are obtainable from a peptide as described above as a starting molecule, for example from a molecule with one of the amino acid sequences according to SEQ ID NO: 1-32, on which, for example, one or more amino acid substitutions, including single or multiple conservative amino acid substitution, were carried out, the resulting peptide being at least 80% and increasingly specifically at least 81%, 82%, 83%, 84%, 85%, 86%, 87%, 88%, 89%, 90%, 91%, 92%, 93%, 94%, 95%, 96%, 97%, 98%, 99% and less than 100% sequence identity with one of the amino acid sequences according to SEQ ID NO: 1-32.

The term “conservative amino acid substitution” means the exchange (substitution) of an amino acid residue for another amino acid residue, whereby this exchange does not lead to a change in polarity or charge at the position of the exchanged amino acid, e.g. the exchange of a non-polar amino acid residue for another non-polar amino acid residue. Conservative amino acid substitutions within the scope of the disclosure include, for example: G=A=S, I=V=L=M, D=E, N=Q, K=R, Y=F, S=T, G=A=I=V=L=M=Y=F=W=P=S=T. However, in certain embodiments such exchanges do not have glycine or tyrosine as the target amino acid or, for example, no amino acid that has a low alpha-helix-forming potential.

In certain embodiments, the peptide according to the disclosure can also be modified. Modifications can be, for example, coupling the peptide with certain other molecules or chemical groups, for example organic (macro) molecules, for example via a covalent bond or a linker/spacer via a suitable amino acid of the chain and/or N- and/or C-terminal.

All of the aforementioned features and embodiments can be implemented individually or in any combination.

Furthermore, the peptide according to the disclosure can also be at least one subunit (module) of a larger peptide or polypeptide, where the polypeptide can comprise a multimer of the sequences described herein, for example 1 to 30 repeats, more specifically 2 to 15 repeats, particularly specifically 2 to 10 repeats, e.g. 2, 3, 4, 5 or 6 repeats of the peptide. The polypeptide may include or consist of such multimers. The term “polypeptide” in this context refers in particular to those peptides that comprise 100 or more amino acids. The term “larger peptides” specifically refers to peptides with at least 40 amino acids, unless otherwise described.

In various embodiments, the peptide is a peptide or polypeptide (multimer) comprising two or more of the peptides as described herein. In various embodiments, the two or more peptides can be connected to one another by at least one linker/spacer, specifically the at least one linker comprises or consists of 1 to 10 amino acid residues, in particular 2, 3 or 4 amino acid residues, specifically selected from the group consisting of G, P, I, A and S or combinations thereof, in particular GPI or GAS. In such embodiments, the individual peptides are optionally connected linearly to one another via peptide bonds, possibly also via a linker.

The peptides described herein may have been chemically synthesized in various embodiments and/or recombinantly produced using protein design. Nowadays, short peptides can easily be prepared synthetically, for example using solid-phase synthesis such as Merrifield's solid-phase synthesis. Longer peptides and polypeptides, on the other hand, are often produced recombinantly in the host organism, e.g. in bacteria or yeast.

The peptides and/or peptide conjugates according to the disclosure can be prepared using recombinant processes. This includes all genetic engineering or microbiological processes that are based on the genes for the peptides of interest being introduced into a host organism suitable for production and transcribed and translated by it (summarized in the context of this disclosure as biotechnological processes).

The peptides and/or peptide conjugates according to the disclosure are particularly specifically produced as polypeptides (multimers) and subsequently cleaved into the functional peptides and/or peptide conjugates. Very particularly the multimers have 1 to 30 peptide units (each according to the disclosure), each of which is separated from one another by linkers of 1 to 10 amino acids long (e.g. 1, 2, 3 or 4 amino acids). Alternatively, the linkers can also be or include interfaces for specific proteases/peptidases, in particular endopeptidases, or can form such an interface together with parts of the peptide.

Using methods that are generally known today, such as chemical synthesis or the polymerase chain reaction (PCR) in conjunction with standard molecular biological and/or protein chemical methods, it is possible for a person skilled in the art to identify the corresponding nucleic acids and even complete genes using known DNA and/or amino acid sequences to produce.

In specified embodiments, the peptide and/or peptide conjugate described herein is produced using biotechnological processes as described above. The at least one peptide and/or peptide conjugate according to the disclosure is suitable for adhesion and/or binding to textiles, as described herein.

In various embodiments, the peptide and/or peptide conjugate described herein is, but is not limited to, are included in the color care rinse aid composition according to the disclosure in a concentration of about 0.00001 to about 5 wt % by weight of the color care rinse aid composition, specifically in a concentration of about 0.0001 to about 2 wt %, more specifically about 0.001 to about 1 wt %, and yet more specifically about 0.01 to about 0.1 wt %.

The color care fabric rinse aid composition comprising the chlorine scavenging peptide is in liquid form. The color care fabric rinse aid composition comprises a liquid diluent, a chlorine scavenging peptide, and optionally an additional laundry care ingredient. The chlorine scavenging peptide can further be a textile-binding peptide as described herein. The color care fabric rinse aid composition can optionally further comprise an antimicrobial agent, a colorant, an electrolyte, an enzyme, a free fragrance, an encapsulated fragrance, an organic solvent, a pH adjusting agent, a preservative, a secondary chlorine scavenger, a solubilizer, a surfactant, or a combination thereof. In certain embodiments, the liquid diluent is water.

The liquid diluent of the rinse aid composition can be water or an organic solvent, or a combination thereof. Suitable organic solvents include an alcohol (e.g. mono, di, or polyhydric alcohols), an ether (e.g., glycol ethers), or other water miscible organic solvent. Exemplary organic solvents for use in the rinse aid composition include ethanol, n-propanol, i-propanol, a butanol, ethylene glycol, propylene glycol, a butanediol, glycerol, diglycol, propyl diglycol, butyl diglycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl ether, propylene glycol ethyl ether, propylene glycol propyl ether, dipropylene glycol methyl ether, dipropylene glycol ethyl ether, methoxytriglycol, ethoxytriglycol, butoxytriglycol, 1-butoxyethoxy-2-propanol, 3-methyl-3-methoxybutanol, propylene glycol t-butyl ether, or a combination thereof. In certain embodiments, the liquid diluent is aqueous.

The color care rinse aid composition comprises a liquid diluent in an amount of about 50 to about 98 weight percent (wt %), specifically about 60 to about 96 wt %, more specifically about 70 to about 95 wt %, and still more specifically about 80 to about 93 wt % based on the total weight of the color care rinse aid composition.

The color care rinse aid composition can further comprise a fragrance, either in free form, in encapsulated form, or a combination thereof. Fragrances refer to and include any fragrant substance or mixture of substances including natural (obtained by extraction of flowers, herbs, leaves, roots, barks, wood, blossoms or plants), artificial (mixture of natural oils or oil constituents), and synthetically produced odoriferous substances. Suitable fragrances or perfumes can be an ester, an ether, an aldehyde, a ketone, an alcohol, a hydrocarbon, or a combination thereof.

Fragrances may be provided in encapsulated form or a pure, neat oil form. Suitable fragrance oil suitable can be an ester, an ether, an aldehyde, a ketone, an alcohol, a hydrocarbon, and a combination thereof.

The term “encapsulated fragrance” refers to a fragrance entrapped in a microcapsule. The weight of an encapsulated fragrance is the weight of the fragrance entrapped in the microcapsule, not including the weight of the microcapsule. The terms “capsule”, “microcapsule”, and “encapsulate” can be used interchangeably. The term “fragrance encapsulate” refers to a core-shell structure which comprises a microcapsule (shell) and a fragrance (core) entrapped in the microcapsule. The weight of a fragrance encapsulate is the sum of the weight of the microcapsule and the weight of the fragrance (core) entrapped in the microcapsule.

The term “free fragrance” means fragrance which is not encapsulated, which is also used interchangeably with the term “neat fragrance” or “fragrance oil.”

Fragrances may also be provided in an encapsulated form to provide sustained release of the fragrances entrapped in microcapsules. The term “encapsulate” is used to describe a method of protecting a fragrance core. Free fragrance may be either a spray-dried emulsion of discrete microdroplets or a fragrance core surrounded by a polymerized outer coating (capsule or shell) which is impervious to the materials in the fragrance core and the materials which may come in contact with the outer surface of the shell. Encapsulated fragrance can also be described as microcapsules or nanocapsules having a core-shell structure.

In some embodiments, the encapsulated fragrance comprises a fragrance entrapped in a microcapsule having a melamine/formaldehyde shell or a melamine acrylate/acrylamide shell. In other embodiments, the encapsulated fragrance comprises a fragrance spray dried and entrapped in a starch-based material. The fragrance in the fragrance encapsulate can be an ester, an ether, an aldehyde, a ketone, an alcohol, a hydrocarbon, or a combination thereof. The encapsulated fragrance provides a sustained release of fragrance to the color care, fragrance booster composition.

An encapsulated fragrance slurry refers to a suspension or a solution comprising encapsulated fragrances and water. In some embodiments, the encapsulated fragrance slurry comprises about 0.01 to about 20 wt %, specifically about 0.02 to about 15 wt %, more specifically about 0.03 to about 10 wt %, and yet more specifically about 0.04 to about 5 wt % based on the total weight of the color care composition.

The free fragrance can be, for example, an ester, an ether, an aldehyde, a ketone, an alcohol, a hydrocarbon, or a combination thereof, which may be in the form of an oil. The free fragrance oil allows an immediate release of the fragrance. In certain embodiments, the free fragrance oil can be present in an amount of greater than 0% to about 10 wt %, specifically about 0.1% to about 8%, more specifically about 0.5 to about 5 wt %, and yet more specifically about 1% to about 3% based on the total weight of the color care composition.

The free fragrance provides an immediate release of a pleasing odor, while the encapsulated fragrance provides a sustained release of a pleasing odor. Together, the free and encapsulated fragrances determine the total amount of fragrances and a release profile of the composition.

In some embodiments, the total amount of fragrances, provided by free fragrance and encapsulated fragrance, in the color care composition can be, for example, about 0.01% to about 15 wt %, specifically about 0.5 to about 10 wt %, more specifically about 1.0 to about 8 wt %, and still more specifically about 1.5 to about 5 wt % based on the total weight of the color care composition.

In certain embodiments, an organic solvent can be used as a carrier for the free fragrance.

In certain embodiments, the color care composition does not comprise a colorant.

In other embodiments, the color care composition comprises a colorant.

The color of the colorant is not limited, and can be, for example, red, orange, yellow, green, blue, indigo, violet, or a combination thereof.

The colorant can include water-soluble or water-insoluble dye.

When used, the colorant can be present in an amount of greater than 0 to about 0.15 wt %, specifically about 0.0001 to about 0.10 wt %, and more specifically about 0.005 to about 0.5 wt % based on the total weight of the color care composition.

In certain embodiments, the color care composition comprises an electrolyte. Suitable electrolytes include inorganic salts, for example, an alkali metal salt, an alkaline earth metal salt, or a combination thereof. Alkali metal salt can include, for example, salts of lithium, sodium, potassium, or a combination thereof. Suitable counterions include, for example, a halide such as fluoride, chloride, bromide, or iodide; a sulfate, a bisulfate; a phosphate, a monohydrogen phosphate, a dihydrogen phosphate; a carbonate, a monohydrogen carbonate; acetate, citrate, lactate, pyruvate, silicate, ascorbate, or a combination thereof. In certain embodiments, the electrolyte is sodium chloride or magnesium chloride.

In certain embodiments, the color care composition can comprise a secondary chlorine scavenger in combination with the peptide. A secondary chlorine scavenger can include an amine compound such as monoethanolamine (primary amine), a chelant such as iminodisuccinic acid (IDS, secondary amine), ethylenediamine-N,N′-disuccinic acid (EDDS, secondary amine), and the like.

In certain embodiments, the color care composition can comprise an antimicrobial active against bacteria, fungus, a combination thereof, and the like. An exemplary antimicrobial includes a benzalkonium chloride.

In certain embodiments, the color care rinse aid composition can further comprise a pH adjusting agent. Exemplary pH adjusting agents include basic compounds, including alkali or alkaline earth metal hydroxides; and acidic compounds including inorganic or organic acids, such as acetic acid, citric acid, formic acid, malic acid, tartaric acid, and the like. Buffer systems including a weak acid and its conjugate base such as an alkali or alkaline earth metal salt thereof can be used to stabilize the pH of the composition. The organic acids and corresponding salts can have further functions beyond adjusting the pH.

In certain embodiments, the color care rinse aid composition can further comprise a surfactant. The surfactant may be an anionic surfactant, a nonionic surfactant, a cationic surfactant, an ampholytic surfactant, a zwitterionic surfactant, or a combination thereof. In specific embodiments the surfactant is a nonionic surfactant. Suitable nonionic surfactants include, for example, alkoxylated alcohols, polyoxyalkylene alkyl ethers, polyoxyalkylene alkylphenyl ethers, polyoxyalkylene sorbitan fatty acid esters, polyoxyalkylene sorbitol fatty acid esters, polyalkylene glycol fatty acid esters, alkyl polyalkylene glycol fatty acid esters, polyoxyethylene polyoxypropylene alkyl ethers, polyoxyalkylene castor oils, polyoxyalkylene alkylamines, glycerol fatty acid esters, alkylglucosamides alkylglucosides, alkylamine oxides, or a combination thereof. In certain embodiments the nonionic surfactant is an alcohol ethoxylate (AE).

The AE may be a primary and secondary alcohol ethoxylate, specifically a C₈-C₂₀ aliphatic alcohol ethoxylated with an average of from 1 to 20 moles of ethylene oxide per mole of alcohol, and more specifically a C₁₀-C₁₅ primary and secondary aliphatic alcohol ethoxylated with an average of from 1 to 10 moles, or from 3 to 8 moles of ethylene oxide per mole of alcohol.

Exemplary AEs are the condensation products of aliphatic C₈-C₂₀, specifically C₈-C₁₆ primary or secondary, linear or branched chain alcohols with ethylene oxide. In some embodiments, the alcohol ethoxylates contain 1 to 20, or 3 to 8 ethylene oxide groups, and may optionally be end-capped by a hydroxylated alkyl group.

In one embodiment, the AE has Formula (I):

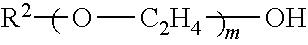

wherein R² is a hydrocarbyl group having 8 to 16 carbon atoms, 8 to 14 carbon atoms, 8 to 12 carbon atoms, or 8 to 10 carbon atoms; and m is from 1 to 20, or 3 to 8. The hydrocarbyl group may be linear or branched, and saturated or unsaturated. In some embodiments, R² is a linear or branched C₈-C₁₆ alkyl or a linear group or branched C₈-C₁₆ alkenyl group, Specifically, R² is a linear or branched C₈-C₁₆ alkyl, C₈-C₁₄ alkyl, or C₈-C₁₀ alkyl group. In the case where the materials contain a range of carbon chain lengths, these carbon numbers represent an average. The alcohol may be derived from natural or synthetic feedstock. In one embodiment, the alcohol feedstock is coconut, containing predominantly C₁₂-C₁₄ alcohol, and oxo C₁₂-C₁₅ alcohols.

In certain embodiments, the color care rinse aid composition can further comprise an enzyme. Suitable enzymes include those from the classes of hydrolases such as proteases, esterases, lipases or lipolytically active enzymes, amylases, cellulases or other glycosyl hydrolases, or a combination thereof. Hydrolases help to remove stains such as protein, fat or starchy stains and graying in the laundry, By removing pilling and microfibrils, cellulases and other glycosyl hydrolases can also help preserve color and increase the softness of the fabric. Oxireductases can also be used to bleach or inhibit the transfer of color. Enzymes obtained from bacterial strains or fungi such as Bacillus subtilis, Bacillus licheniformis, Streptomyceus griseus and Humicola insolens are suitable. Proteases of the subtilisin type and proteases which are obtained from Bacillus lentus can be used. Cutinases are examples of lipolytically active enzymes. Peroxidases or oxidases may be suitable in some cases. Suitable amylases include alpha-amylases, iso-aniylases, pullulanases and pectinases, Cellobiohydrolases, endoglucanases and alpha-glucosidases, which are also called cellobiases, or a combination thereof, can be used as cellulases. The enzymes can be adsorbed on carriers or embedded in coating substances to protect them against premature decomposition. The amount of the enzymes, enzyme mixtures, or enzyme granules can be, for example, about 0.1 to about 5 wt %, specifically about 0.12 to about 2 wt % based on the total weight of the color care composition.

The color care composition can be prepared by a process of mixing a liquid diluent, a chlorine scavenging peptide, and optionally an additional laundry care ingredient together to form a liquid composition. The optional color care composition ingredients can be an antimicrobial agent, a colorant, an electrolyte, an enzyme, a free fragrance, an encapsulated fragrance, an organic solvent, a pH adjusting agent, a preservative, a secondary chlorine scavenger, a solubilizer, a surfactant, or a combination thereof. Certain ingredients can be premixed before being combined with the liquid diluent and other ingredients.

The color care rinse aid composition is liquid at room temperature (e.g., about 25° C.) and miscible in cold, warm, and hot water.

In certain embodiments, the color care fabric rinse aid composition is transparent.

In certain embodiments, the color care fabric rinse aid composition is free of fragrance, perfumes, and dyes.

The color care composition can be incorporated in other laundry care and home care products.

The color care rinse aid composition can be used to treat fabrics or textiles, e.g., to scavenge chlorine from a rinse cycle. A method of scavenging chlorine comprises contacting the fabric or textile with a color care rinse aid composition of the present disclosure. In one embodiment, the subject to be treated is a textile or a fabric.

In certain embodiments, the color care rinse aid composition is used in a rinse cycle after the fabric or textile washing or as the sole agent for treating the laundry, e.g. can be used to refresh the laundry.

The color care rinse aid composition can be added as textile treatment agents manually or in the last rinse cycle when metering in a fabric softener chamber of a washing machine. If used as the sole agent, it can be used either in the hand wash basin or in the washing machine directly in the detergent dispenser for the main detergent.

The color care rinse aid compositions may be used in the rinse cycle of a domestic laundry process. The color care composition can be used in a top loading or front loading washer, in hot, warm, or cold water. The composition may be added directly in an undiluted state to a washing machine, e.g. through a dispenser drawer or, for a top-loading washing machine, directly into the drum. Alternatively, it can be diluted prior to use. The compositions may also be used in a domestic hand-washing laundry operation. In one embodiment, a fabric or textile is treated with a composition as disclosed herein.

In a further embodiment, a method of scavenging chlorine from a wash cycle and a rinse cycle comprises contacting a fabric or textile with the color care composition as described herein in a laundry wash cycle, rinse cycle, or a combination thereof.

In a further embodiment, a method of preventing the deterioration of dyes in a fabric or textile comprises contacting a fabric or textile with the color care composition as described herein in a laundry wash cycle, rinse cycle, or a combination thereof.

In a further embodiment, a method of preventing color loss from a fabric or textile comprises contacting a fabric or textile with the color care composition as described herein in a laundry wash cycle, rinse cycle, or a combination thereof.

The following examples are merely illustrative of the invention disclosed herein and are not intended to limit the scope thereof.

### EXAMPLES

### Example 1. Peptides

Disclosed in Table 1. are peptides including linker/spacer sequences, chlorine scavenging peptides, and textile binding and chlorine scavenging peptides. Suitable linker/spacer sequences include GGGGS, EAAAK, and repeated units thereof. Those peptides which have domains that show affinity to bind to different types of textiles include those that contain one, two, or more amino acids which are susceptible to oxidation (such as Methionine, Lysine, Cysteine, and others). Chlorine scavenging peptides include those that contain one, two, or more amino acids such as Lysine, Threonine, and the like.

TABLE 1
Domain Sequences

| SEQ ID NO: | Peptide sequence | Function |
|---|---|---|
| 01 | GGGGS | Linker |
| 02 | GGGGSGGGGS | Linker |
| 03 | GGGGSGGGGSGGGGS | Linker |
| 04 | EAAAKEAAAKEAAAK | Linker |
| 05 | NGLLIPQFLVASGGGGSRSIVTF | Linker |
| | SLRQNRGGGGSNGLLIPQFLAS | |
| 06 | NGLLIPQFLVASGGGGSRALQAL | Linker |
| | RALQALEALGGGGSNGLLIPQFL | |
| | VAS | |
| 07 | NGLLIPQFLVASGGGGSRALRAL | Linker |
| | QALEALEALGGGGSNGLLIPQFL | |
| | VAS | |
| 08 | RSIVTFSLRQNR | Linker |
| 09 | RALQALRALQALEAL | Linker |
| 10 | RALRALQALEALEAL | Linker |
| 11 | NGLLIPQFLVASGGGGSMSDYQM | Linker |
| | DM | |
| 12 | RALRALQALEALEALGGGGSMSD | Linker |
| | YQMDM | |
| 13 | RALQALRALQALEALGGGGSMSD | Linker |
| | YQMDM | |
| 14 | NGLLIPQFLVASGGGGSMDMQGR | Linker |
| | YMDR | |
| 15 | RALRALQALEALEALGGGGSMDM | Linker |
| | QGRYMDR | |
| 16 | RALQALRALQALEALGGGGSMDM | Linker |
| | QGRYMDR | |
| 17 or P8- | MYMDMKDVMMKYDMAMMEMKMDS | Artificial |
| Scavenger-1 | GGGRSIVTFSLRQNRGGGSMYMD | Peptide |
| | MKDVMMKYDMAMMEMKMD | |
| 18 or P8- | MYMDMKDVMMKYDMAMMEMKMDR | Artificial |
| Scavenger-2 | SIVTFSLRQNRMYMDMKDVMMKY | Peptide |
| | DMAMMEMKMD | |
| 19 or P8- | MKMYMKMDRSIVTFSLRQNRMKM | Artificial |
| Scavenger-3 | YMKMD | Peptide |
| 20 or P8- | MMYMMYMRSIVTFSLRQNRMMYM | Artificial |
| Scavenger-4 | MYM | Peptide |
| 21 or P8- | NGLLIPQFLVASGGGGSRSIVTF | Artificial |
| SRP | SLRQNRGGGGSNGLLIPQFLVAS | Peptide |
| 22 or P8- | KYGDMKDVKMKYDKACSEMKYDS | Artificial |
| Scavenger-5 | GGGRSIVTFSLRQNRGGGSKYGD | Peptide |
| | MKDVKMKYDKACSEMKYD | |
| 23 or P8- | KYGDMKDVKMKYDKACSEMKYDR | Artificial |
| Scavenger-6 | SIVTFSLRQNRKYGDMKDVKMKY | Peptide |
| | DKACSEMKYD | |
| 24 | GGGGS | Linker |
| 25 | GGGGSGGGGS | Linker |
| 26 | GGGGSGGGGSGGGGS | Linker |
| 27 | GGGGSGGGGSGGGGSGGGGS | Linker |
| 28 | GGGGGGGG | Linker |
| 29 | GGGGGG | Linker |
| 30 | EAAAK | Linker |
| 31 | EAAAKEAAAK | Linker |
| 32 | EAAAKEAAAKEAAAK | Linker |
| 33 | AEAAAKEAAAKEAAAKEAAAKAL | Linker |
| | EAEAAAKEAAAKEAAAKEAAAKA | |
| 34 | PAPAP | Linker |
| 35 | AEAAAKEAAAKA | Linker |

### Example 2. Color Care Rinse Aid Formulations

Table 2 below describes exemplary Color Care Rinse Aid Formulations comprising peptides to scavenge chlorine in the rinse phase for fabric care benefit.

TABLE 2

| | Formula (wt %) | | | | | | | |
|---|---|---|---|---|---|---|---|---|
| Ingredient | 1 | 2 | 3 | 4 | 5 | 6 | 7 | 8 |
| 50% Citric Acid | 46.85 | 46.85 | 46.85 | | | 1 | 1 | 1 |
| Solution | | | | | | | | |
| 14.7% Acetic | 0 | 0 | 0 | | | | | |
| Acid Solution | | | | | | | | |
| 30% Sodium | 0.5 | 0.5 | 0.5 | | | | | |
| Formate Solution | | | | | | | | |
| 50% Sodium | 3 | 3 | 3 | | | | | |
| Hydroxide | | | | | | | | |
| Propylene Glycol | 5 | | | | | | | |
| Glycerin | | 7 | | | | | | |
| PEG 400 | | | 5 | | | | | |
| Sodium | 2.22 | 2.22 | 2.22 | | | | | |
| Cumensulfonate | | | | | | | | |
| 25-7 Nonionic | 2.5 | 2.5 | 2.5 | 3 | 5 | 5 | 5 | 5 |
| Alcohol | | | | | | | | |
| Ethoxylate | | | | | | | | |
| Sodium | | | | | | 10 | 15 | 20 |
| Carbonate | | | | | | | | |
| Perfume | 2 | 2 | 2 | 7 | 7 | 5 | 5 | 5 |
| Encapsulated | | | | 2 | 2 | | | |
| Fragrance | | | | | | | | |
| Peptide (50% | 0.1 | 0.1 | 0.1 | 0.1 | 0.1 | 0.1 | 0.1 | 0.1 |
| solution) | | | | | | | | |
| Water | to | to | to | to | to | to | to | to |
| | 100% | 100% | 100% | 100% | 100% | 100% | 100% | 100% |
| pH Target | 2.3 to | 2.3 to | 2.3 to | 6 to 8 | 6 to 8 | 10.3 to | 10.3 to | 10.3 to |
| | 2.9 | 2.9 | 2.9 | | | 11.3 | 11.3 | 11.3 |

Table 3. below describes additional exemplary Color Care Rinse Aid Formulations comprising different concentrations of peptides to scavenge chlorine in the rinse phase for fabric care benefit.

TABLE 3

| | 1.5% Active Peptide | | 0.5% Active Peptide | | 0.1% Active Peptide | |
|---|---|---|---|---|---|---|
| | A | B | C | D | E | F |
| Water | 35.335 | 36.09813 | 36.075 | 36.21375 | 36.371 | 36.39875 |
| Peptide Powder | 0 | 1 | 0 | 0.5 | 0 | 0.1 |
| (100% active) | | | | | | |
| Glycerin | 60.165 | 61.46438 | 61.425 | 61.66125 | 61.929 | 61.97625 |
| Peptide; 50% | 3 | 0 | 1 | 0 | 0.2 | 0 |
| Solution | | | | | | |
| IDS 34% Solution | 1.5 | 1.5 | 1.5 | 1.5 | 1.5 | 1.5 |
| Total (wt %) | 100 | 100 | 100 | 100 | 100 | 100 |
| Water Activity | 0.7052 | 0.6932 | 0.7055 | 0.696 | 0.6995 | 0.6979 |
| Viscosity | 13 | 12 | 15 | 12 | 15 | 12 |
| pH | 9.99 | 8.88 | 10.18 | 9.08 | 10.54 | 10.23 |

TABLE 4

| | Chlorine ppm | | | | |
|---|---|---|---|---|---|
| | | 15 | 30 | 1 | 2 |
| Molar Equivalence | Start | Seconds | Seconds | Minute | Minute |
| 0.14% NaOH | 1 | 1 | | 1 | 1 |
| (Control) | | | | | |
| 0.25% L-Lysine | 1 | 0 | | 0 | 0 |
| 0.1% | 1 | 0 | | 0 | 0 |
| Monoethanolamine | | | | | |
| 0.14% NaOH | 3 | 3 | | 3 | 3 |
| (Control) | | | | | |
| 0.25% L-Lysine | 3 | 0 | | 0 | 0 |
| 0.1% | 3 | 1 | | 1 | 1 |
| Monoethanolamine | | | | | |
| Formulation F | 3 | 2 | 1.5 | 1 | 1 |
| Formulation E* | 3 | 0 | 0 | 0 | 0 |
| (Lysine HCl) | | | | | |

Contains Lysine HCl as a stand in for peptide

The Formulation E and F were tested for chlorine scavenging capability. The target dose of detergent per wash is approximately 45 grams and the estimated amount of water in a High Efficiency Washing Machine is 31.4 L. Proportionally, 1.4 grams of each formula will go into 1 L of water (benchtop scale) at the disclosed ppm level of hypochlorite.

The above formulations in Table 3. were created by adding the stated level of active material (NaOH, L-Lysine, or Monoethanolamine) and then adjusting the pH to 8 with Citric Acid or NaOH. Each of the active materials were equivalent on a molar basis. The L-Lysine samples serve as a proxy for a lysine-rich peptide. The results of the chlorine scavenging capability test in Table 4. demonstrates that L-Lysine is an effective chlorine scavenger, and can outperform Monoethanolamine on a molar basis.

### Example 3. Small Scale Evaluation of Chlorine Scavenging Carry-Over

Protocol: The following describes a small scale method for evaluating the efficacy of peptide reagents for binding to fabric and scavenging free chlorine in a simulated wash and rinse cycle.

A 400 mL glass beaker equipped with a cross-shaped stir bar is used as the washing/rinsing vessel. Two hundred grams of prepared water is weighed into the beaker. The water is prepared with 120 ppm hardness (calcium and magnesium, 3:1 ratio), and 3 ppm free chlorine via a bleach solution. The free chlorine level is monitored using an Insta-Test Analytic free chlorine test strip from LaMotte. The wash water is heated on a hot plate to 90° F. (32.2° C.).

The chemistry of interest is injected into the wash water via a micropipette. For the reported tests, a 1% by weight solution of selected peptide in deionized water is prepared and injected into the wash water to deliver 20 mg of active ingredient (2 mL). For P8-Scavenger 4, a 0.5% by weight solution was prepared in a 1:1 Water:Ethanol mixture and dosed appropriately. The peptide solution is stirred for 1 minute.

Pre-cut cotton/polyester fabric sheets (55/45 ratio from SDL Atlas) weighed out to 4 grams are added directly to the wash water. The water, peptide, and ballast mixture are allowed to stir for 12 minutes. Afterwards, the ballast is removed from the water and placed in a device which applies centrifugal force to remove excess water from the sample. The ballast is spun out for about 2 minutes.

Separately, a new 400 mL beaker is prepared with 200 grams of water (120 ppm hardness, 3 ppm free chlorine) to simulate the rinse cycle. The temperature is set to 60° F. (about 15.5° C.). The free chlorine level is checked with a test strip. The treated ballast is added to the rinse water and stirred for 4 minutes. The chlorine scavenging rate is monitored by using the free chlorine test strips at various time points (e.g. 2 minutes, 4 minutes).

Results: The procedure was conducted using polylysine (>95% purity) from MarkNature as a control, as well as the specialized peptide constructs. Table 5. below shows the free chlorine level (FCL) in the simulated rinse water over the course of 4 minutes, as determined based on the visual indicator test strips.

TABLE 5

| | | | FCL in | FCL in | FCL in | FCL in |
|---|---|---|---|---|---|---|
| | Amino | FCL in | Rinse | Rinse | Rinse | Rinse |
| Sample | Acid | Rinse | (1 | (2 | (3 | (4 |
| Identify | Sequence | (Initial) | minute) | minutes) | minutes) | minutes) |
| Poly-lysine | — | 3.0 ppm | 2.0 ppm | 1.0 ppm | 0.5 ppm | 0.25 ppm |
| P8- | MKMYMK | 3.0 ppm | 1.5 ppm | 0.25 ppm | 0 ppm | 0 ppm |
| Scavenger-3- | MDRSIVTFS | | | | | |
| SEQ. ID. | LRQNRMK | | | | | |
| NO: 19 | MYMKMD | | | | | |
| P8- | MMYMMY | 3.0 ppm | 2.0 ppm | 1.0 ppm | 0.5 ppm | 0.25 ppm |
| Scavenger-4- | MRSIVTFSL | | | | | |
| SEQ. ID. | RQNRMMY | | | | | |
| NO: 20 | MMYM | | | | | |
| P8-SRP- | NGLLIPQFL | 3.0 ppm | 2.5 ppm | 2.0 ppm | 1.5 ppm | 1.0 ppm |
| SEQ. ID. | VASGGGGS | | | | | |
| NO: 21 | RSIVTFSLR | | | | | |
| | QNRGGGGS | | | | | |
| | NGLLIPQFL | | | | | |
| | VAS | | | | | |

It was observed that P8-Scavenger 3 was highly effective for scavenging free chlorine in the simulated rinse water compared to polylysine, indicating effective carry-over on the ballast fabric. P8-Scavenger-4 provided similar chlorine scavenging efficacy to polylysine, whereas P8-SRP only provided modest scavenging of free chlorine in the rinse water. The peptide sequence has a significant impact on its scavenging efficacy in simulated rinse water.

### Example 4: Color Care Rinse Aid Formulations

Table 6. below describes additional exemplary Color Care Rinse Aid Formulations comprising different concentrations of peptides to scavenge chlorine in the rinse phase for fabric care benefit.

TABLE 6

| | Formula G | Formula H | Formula I | Formula J | Formula K | Formula L |
|---|---|---|---|---|---|---|
| Ingredient | Active % | Active % | Active % | Active % | Active % | Active % |
| Deionized Water | Balance | Balance | Balance | Balance | Balance | Balance |
| | (~65.2) | (~65.2) | (~65.1) | (~65.1) | (~65.1) | (~65.1) |
| Citric Acid | 25.00 | 25.00 | 25.00 | 25.00 | 10 to 40 | 10 to 40 |
| Sodium Hydroxide | 2.32 | 2.32 | 2.32 | 2.32 | 1 to 5 | 1 to 5 |
| Cocamidopropyl | 3.73 | 0 | 3.73 | 0 | 1 to 8 | 0 |
| Betaine | | | | | | |
| Cocamidopropyl | 0 | 3.73 | 0 | 3.73 | 0 | 1 to 8 |
| Hydroxysultaine | | | | | | |
| Sodium Xylene | 2.00 | 2.00 | 2.00 | 2.00 | 0.5 to 5 | 0.5 to 5 |
| Sulfonate | | | | | | |
| Fragrance (Neat | 1.50 | 1.50 | 1.50 | 1.50 | 0 to 5 | 0 to 5 |
| and/or | | | | | | |
| Encapsulated) | | | | | | |
| Preservative | 0.25 | 0.25 | 0.25 | 0.25 | 0.05 to 0.5 | 0.05 to 0.5 |
| Peptide (Delivered | 0 | 0 | 0.1 | 0.1 | 0.0001 to 2 | 0.0001 to 2 |
| as a 50% solution) | | | | | | |

The pH target of formula G to L is about 2 to about 5, specifically about 2 to about 4, and more specifically about 2.25 to about 3.25. This desired pH drops the rinse water to an acidic pH, with a target pH less than 5 (about 3.25 to about 3.5). If these formulas are added to the wash cycle, it would drop the wash water liquor down to a pH less than 5 (about 3.25 to about 3.5).

For preservation, Sorbic Acid, Benzoic Acid, Glutaraldehyde and other preservatives commonly known to the art can be used.

For surfactants or stabilizers, other anionic, nonionic, amphoteric, zwitterionic (etc.) can be utilized.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “+10%” means that the indicated measurement can be from an amount that is minus 10% to an amount that is plus 10% of the stated value. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A liquid color care rinse aid composition, comprising
an artificial peptide;
a liquid diluent; and
optionally an additional ingredient,
wherein the artificial peptide is composed of a textile binding domain and a chlorine scavenger domain.

2. The composition of claim 1, wherein the artificial peptide is at least one of SEQ. ID NO: 01-32.

3. The composition of claim 1, wherein the artificial peptide is further composed of a linker domain.

4. The composition of claim 1, wherein the artificial peptide is composed of at least one additional textile binding domain.

5. The composition of claim 1, wherein the peptide is composed of at least one additional chlorine scavenger domain.

6. The composition of claim 1, wherein the chlorine scavenger domain is predominantly composed of at least one of the following amino acids: lysine, arginine, histidine, glutamine, and tryptophan.

7. The composition of claim 1, wherein the textile binding domain is predominantly composed of alpha helices.

8. The composition of claim 1, wherein the textile binding domain is predominantly composed of one or more amino acids selected from the group of: methionine, alanine, leucine, glutamate, and lysine.

9. The composition of claim 1, wherein the liquid diluent is water, an alcohol, an ether, another water miscible organic solvent, or a combination thereof.

10. The composition of claim 1, wherein the liquid diluent is aqueous.

11. The composition of claim 1, wherein the additional ingredient is an antimicrobial agent, a colorant, an electrolyte, an enzyme, a free fragrance, an encapsulated fragrance, an organic solvent, a pH adjusting agent, a preservative, a secondary chlorine scavenger, a solubilizer, a surfactant, or a combination thereof.

12. The composition of claim 11, wherein the secondary chlorine scavenger is iminodisuccinic acid, ethylenediamine-N,N′-disuccinic acid, monoethanolamine, or a combination thereof.

13. The composition of claim 1, further comprising a fragrance.

14. The composition of claim 1, further comprising a free fragrance, an encapsulated fragrance, or a combination thereof.

15. A method of scavenging chlorine from a wash cycle and a rinse cycle; a method of preventing the deterioration of dyes in a fabric or textile; or a method of preventing color loss from a fabric or textile, comprising:
contacting a fabric or textile with the color care composition of claim 1 in a laundry wash cycle, rinse cycle, or a combination thereof.
